(19) United States
(12) Patent Application Publication
(10) Pub. No.: US 2026/0028185 A1
(43) Pub. Date: Jan. 29, 2026

(54) Innovative Diaper Dispensing System with Enhanced Safety and Aesthetic Features

(71) Applicant: Michael J Bryant, Tampa, FL (US)

(72) Inventor: Michael J Bryant, Tampa, FL (US)

(21) Appl. No.: 19/345,826

(22) Filed: Sep. 30, 2025

Related U.S. Application Data
(63) Continuation-in-part of application No. 18/636,629, filed on Apr. 16, 2024.
(60) Provisional application No. 63/618,417, filed on Jan. 8, 2024.

(51) Int. Cl. B65G 1/07; A61F 15/00

(52) U.S. Cl. CPC B65G 1/07; A61F 15/001

(57) ABSTRACT

A storage and dispensing system is provided for efficiently loading, supporting, and dispensing one or more items. The system includes a housing defining an interior space for storing the items, a lift platform assembly disposed within the housing and configured to move vertically to support and elevate the items, and a loading mechanism operatively connected to the lift platform assembly. The loading mechanism facilitates placement or loading of the items onto the lift platform assembly and interacts with the lift platform to transition it between a lowered position for loading and a raised position for dispensing. The system allows for selective dispensing of items while maintaining organized storage and ease of access.

### CROSS-REFERENCE TO RELATED APPLICATIONS

This application is a Continuation-in-Part of U.S. application Ser. No. 18/636,629, filed on Apr. 16, 2024, which claims the benefit of U.S. Provisional Patent Application No. 63/618,417, filed on Jan. 8, 2024.

### BACKGROUND

### Technical Field

The present invention relates generally to storage and dispensing systems, and more particularly, to an improved system for storing and dispensing items, such as diapers, in a manner that enhances convenience, safety, and ease of use through integrated structural and functional components.

### Background Information

The present invention generally relates to dispensing systems for stacked articles and, more particularly, to systems designed to store and dispense individual items such as folded garments, personal care products, or diapers in an organized, sanitary, and efficient manner. Devices for dispensing stacked articles are used in a variety of settings, including households, nurseries, healthcare facilities, and daycares, where quick, reliable access to clean, undamaged items is essential.

In the specific context of diaper changing, dispensing systems play a vital role in helping caregivers manage frequent and repetitive tasks. Changing diapers is a demanding and time-sensitive activity that must often be performed under less-than-ideal circumstances. While several products have been developed to aid in the storage and dispensing of clean diapers, existing systems often fall short in terms of ease of use, reliability, safety, and adaptability to different environments.

For example, U.S. Pat. No. 5,678,727 to Rubin et al. and U.S. Pat. No. 6,817,483 B2 to Finken et al. disclose diaper storage and dispensing devices that provide basic mechanical dispensing functions. However, these systems do not include critical user-focused features such as reliable reloading mechanisms, child-resistant access doors, or configurations that prevent jamming or uneven dispensing of diapers. In some cases, spring mechanisms are exposed or unsecured, presenting potential safety hazards, particularly in homes or facilities where young children are present.

A further drawback of many existing dispensers is the difficulty involved in reloading diaper stacks. Poorly designed loading mechanisms often require users to remove multiple components or struggle with misaligned elements, increasing the time and mess associated with refills. Additionally, conventional devices may compress or deform diapers during use, making them harder to retrieve and reducing their effectiveness.

Other deficiencies include the lack of visual or tactile indicators of low supply, limited configurability for various locations and user preferences, and minimal consideration for aesthetic or ergonomic integration into nursery or home environments. Prior systems also typically lack integrated features or decorative finishes that can improve the overall organization and user experience during diaper changes.

Accordingly, there exists a need for an improved dispensing system for storing and dispensing one or more items, such as diapers, that addresses the foregoing and other shortcomings in the conventional art.

### SUMMARY

The present invention provides a storage and dispensing system configured to efficiently store and dispense stacked items, such as diapers, in a controlled and user-friendly manner. The system includes a housing that defines an interior space and incorporates an access opening to facilitate retrieval of stored items without requiring full access to the housing interior. This configuration improves convenience, cleanliness, and accessibility during repeated daily use.

In one embodiment, the invention features a lift platform assembly disposed within the housing and configured to support and elevate stored items as they are dispensed. The lift platform assembly comprises a vertically movable shelf platform, at least one guide rail secured to an interior wall of the housing, and at least one coil spring mechanism. The coil spring mechanism is operatively coupled to the shelf platform and configured to exert an upward biasing force to elevate the platform, while the guide rail ensures smooth, aligned vertical movement.

In certain embodiments, the guide rail is a single rail mounted to a rear wall of the housing, and the shelf platform includes a notch shaped to receive the guide rail and conform to its cross-section. This design promotes stability and alignment during vertical movement of the platform. The coil spring mechanism includes a coiled spring housed in a spring housing with an extendable portion that unrolls along the guide rail during platform loading and retracts to raise the platform after loading.

In alternative embodiments, the lift platform assembly is supported between two opposing vertically oriented guide rails mounted along interior side walls of the housing. A pair of coil springs housed in respective spring housings cooperate with each guide rail to elevate the platform. The shelf platform includes notches along its lateral edges to receive the guide rails, allowing for clear and stable movement while providing clearance for the spring components.

The system further includes a loading mechanism that temporarily restrains the shelf platform in a lowered position to enable placement of items. Upon release, the spring mechanism returns the platform to an elevated position. The coil springs are configured to store mechanical energy during downward displacement and to release the energy to elevate the platform during dispensing. This eliminates the need for external power sources or manual lifting mechanisms.

Additional embodiments include a visual indicator assembly with an indicator pin actuated by the shelf platform to signal a low or depleted supply of items. Transparent windows may be integrated into the housing to permit visual inspection of the item supply without opening the housing. Further variations include a lockable lid with semicircular extensions to form a sealed upper enclosure, decorative tops, and optional wipe holders mounted to the housing. Configurations other than semicircular, such as rectangular, polygonal, or irregularly curved shapes, may also be suitable for forming a sealed upper enclosure.

These and other features of the invention work collectively to streamline and improve the loading, storage, and dispensing of items, particularly in contexts such as infant care, healthcare, or any environment requiring rapid and sanitary access to stacked products.

### BRIEF DESCRIPTION OF THE DRAWINGS

The foregoing and other features and advantages of the invention will be more fully understood from the following detailed description, taken in conjunction with the accompanying drawings, in which:

FIG. 1 is a front perspective view of a storage and dispensing system (hereinafter also referred to as the “system”) in accordance with an embodiment of the present invention.

FIG. 2 is a front perspective view similar to FIG. 1, illustrating the system with the housing door in an open position.

FIG. 3 is a front view of the system shown in FIG. 2, with the diaper stack removed from the housing.

FIG. 4 is a top view of the system shown in FIG. 3.

FIG. 5 is a partial cross-sectional view of the system taken along line 5-5 of FIG. 1, with the diaper stack removed from the housing.

FIG. 6A is a front perspective view similar to FIG. 2, showing the shelf platform in a lowered position within the housing and coupled to a loop connector for loading a stack of diapers.

FIG. 6B is a front view of the system shown in FIG. 6A.

FIG. 7 is a right-side view of the system shown in FIG. 1.

FIG. 8 is a perspective view showing the assembly of the shelf platform, rail, and spring mechanism, with other components omitted for clarity.

FIG. 9 is an exploded view of the system according to the first embodiment.

FIG. 10 is a front perspective view similar to FIG. 1, further illustrating a lockable lid for the system.

FIG. 11 is a view similar to FIG. 10, showing the lockable lid in an open position.

FIG. 12 is a front view of the system shown in FIG. 10.

FIG. 13A is a front perspective view similar to FIG. 10, further illustrating a connecting member for the removable attachment of a decorative top to the housing.

FIG. 13B is a front perspective view similar to FIG. 13A, showing the decorative top removably connected to the housing.

FIG. 14 is a perspective view showing the assembly of the shelf platform, rails, and spring mechanisms according to another embodiment of the system.

FIG. 15 is a front perspective view similar to FIG. 1, further illustrating a wipe holder removably mounted to the housing.

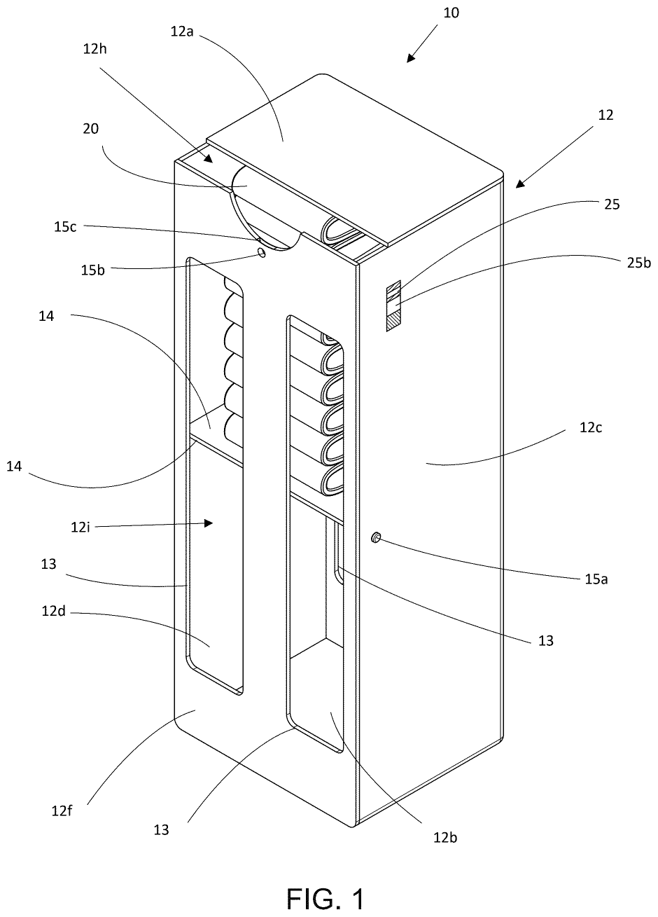

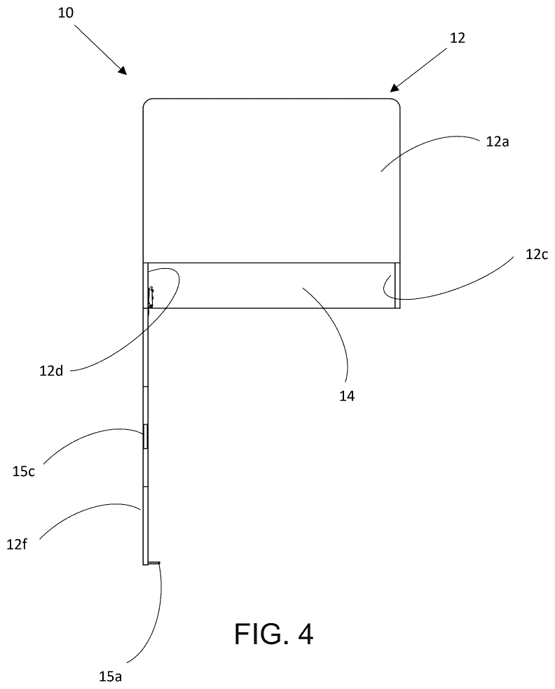

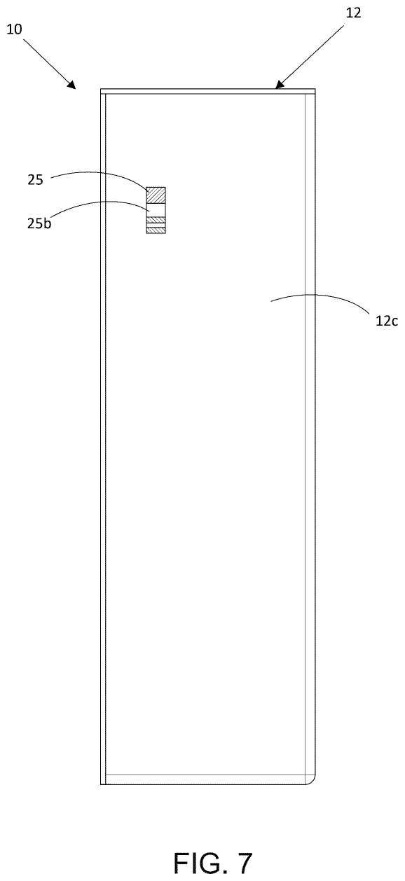

Throughout FIGS. 1-15, identical reference numerals are used to indicate corresponding elements for consistency and ease of understanding.

### DETAILED DESCRIPTION

It should be understood that the following description, including both the general overview and the detailed embodiments, is provided for purposes of illustration and to facilitate a better understanding of the invention, and is not intended to limit the invention as defined by the claims. The accompanying drawings, which form part of this specification, depict representative embodiments and are intended to assist in explaining the principles of the invention. Consistent reference numerals are used throughout the drawings to denote the same elements. Specific language is used in the description to convey the invention's features clearly; however, it is not intended to restrict the scope of the invention, as various modifications, adaptations, and combinations of the described elements will be apparent to those skilled in the art. The system, methods, and examples provided are illustrative and exemplary only, and it should be understood that any feature, structure, or characteristic disclosed herein may be combined or altered in any appropriate manner without departing from the scope and spirit of the invention. Unless stated otherwise, all terminology used carries the meaning commonly ascribed to it by those of ordinary skill in the relevant technical field.

The present invention relates to a storage and dispensing system designed to simplify and improve the process of retrieving stacked items by providing an efficient, safe, sanitary, and convenient method for storage and dispensing. The system combines an innovative spring mechanism with a structured housing to enable easy and organized access to items while minimizing mess and handling effort. The system further offers modular functionality for integrating additional components such as accessory storage and aesthetic customization, enhancing its practicality and adaptability to a range of user needs and environments. In particular, the system is well-suited for dispensing diapers, addressing the frequent and demanding nature of diaper changes faced by parents and caregivers.

Although the system of the present invention is disclosed herein with reference to diaper storage and dispensing, the system is also adapted for use with a wide variety of other stackable or similarly packaged items. These include, but are not limited to, sanitary wipes, adult incontinence products, paper towels, tissues, napkins, disposable gloves, and other hygiene or household products that benefit from structured, sanitary, and user-friendly dispensing. The system's modularity and scalable architecture enable it to be customized for various commercial, residential, or institutional applications.

FIGS. 1-9 illustrate an embodiment of a storage and dispensing system (also referred to herein as the “diaper dispensing system,” “dispensing system,” or simply the “system”), generally designated with reference numeral 10, according to an embodiment of the present invention. The system 10 includes a housing 12 configured to store and elevate a vertical stack of diapers 20 for hygienic and convenient access. The system 10 also incorporates a spring-biased lift platform assembly and a loading mechanism, as further described below. The lift platform assembly maintains the top diaper at an accessible height as the stack depletes, reducing the need to reach into the housing. The loading mechanism facilitates easy reloading of the diaper stack, helping to minimize disruption and maintain cleanliness during refill.

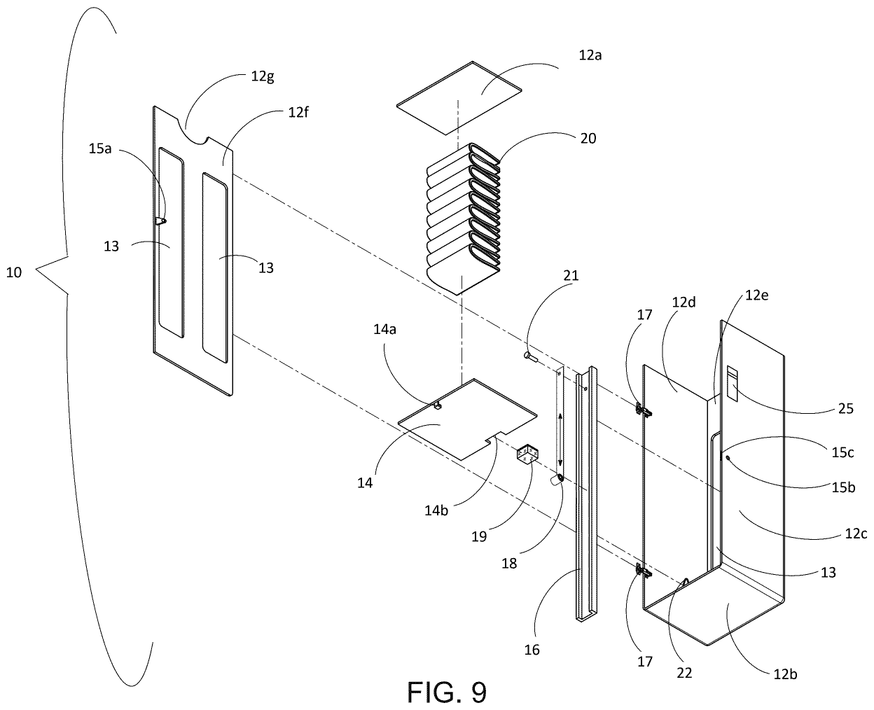

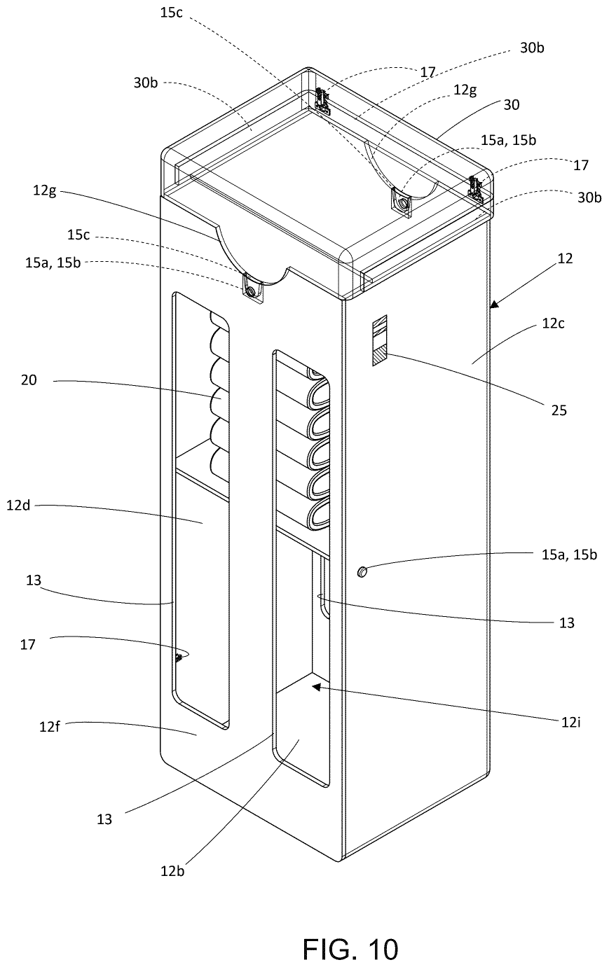

FIG. 9 is an exploded view illustrating the components of the diaper dispensing system 10 according to this embodiment. It should be noted that the stack of diapers 20 shown in FIGS. 1, 2, 6A, 6B, and 9, as well as in the modified embodiments illustrated in FIGS. 10-12, 13A-13B, and 15, is provided for illustrative purposes only. The diapers themselves are not part of the claimed invention and are shown solely to demonstrate the intended use and operation of the system 10.

Referring to FIGS. 1-4, 6A, 6B, 7 and 9, the housing 12 has an interior space 12i for storing the stack of diapers 20 and includes a top wall 12a, a bottom wall 12b, side walls 12c, 12d, a rear wall 12e, and a front-facing hinged door 12f, attached to side wall 12d by hinges 17. The top wall 12a and door 12f form a diaper access opening 12h, enabling the user to remove the top diaper without opening the door fully, minimizing contamination and disruption during diaper changes. A door slot 12g and a corresponding slot 12g formed in the rear wall 12e create a semi-circular pathway that may also interface with a lockable lid extension, as further described below with reference to FIGS. 10-13B.

Referring to FIGS. 1-4, 6A, 6B, 7, and 9, the housing 12 includes an interior space 12i for storing the stack of diapers 20 and is defined by a top wall 12a, a bottom wall 12b, side walls 12c and 12d, a rear wall 12e, and a front-facing hinged door 12f. The top wall 12a is connected to the upper edges of side walls 12c, 12d, and rear wall 12e to form, together with door 12f, a diaper access opening 12h, as shown in FIG. 1. This access opening allows the user to retrieve the top diaper without fully opening the door, helping to minimize contamination and disruption during diaper changes.

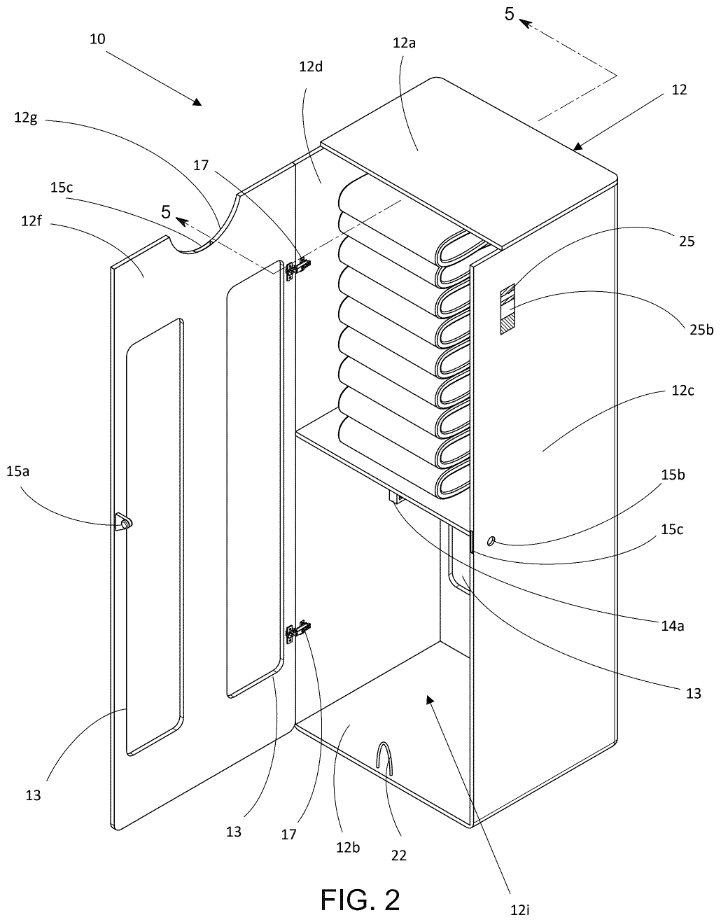

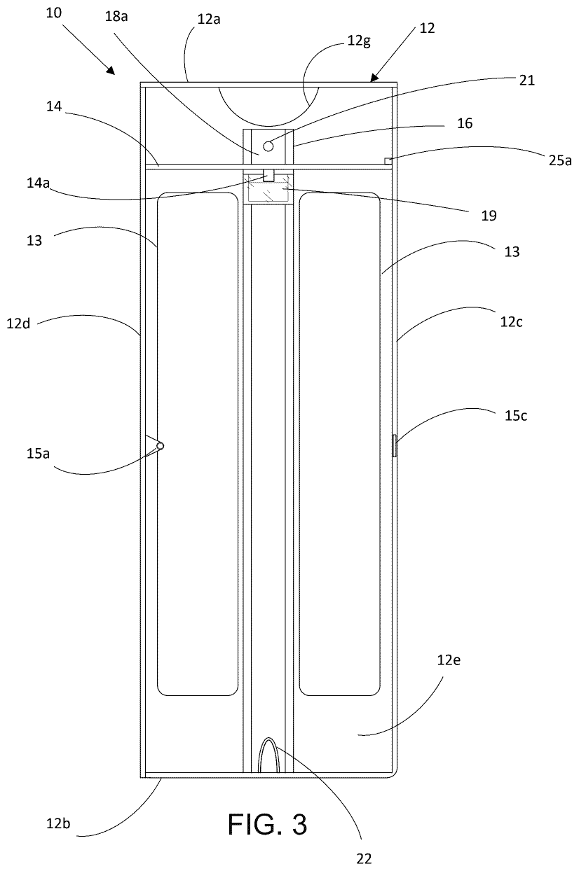

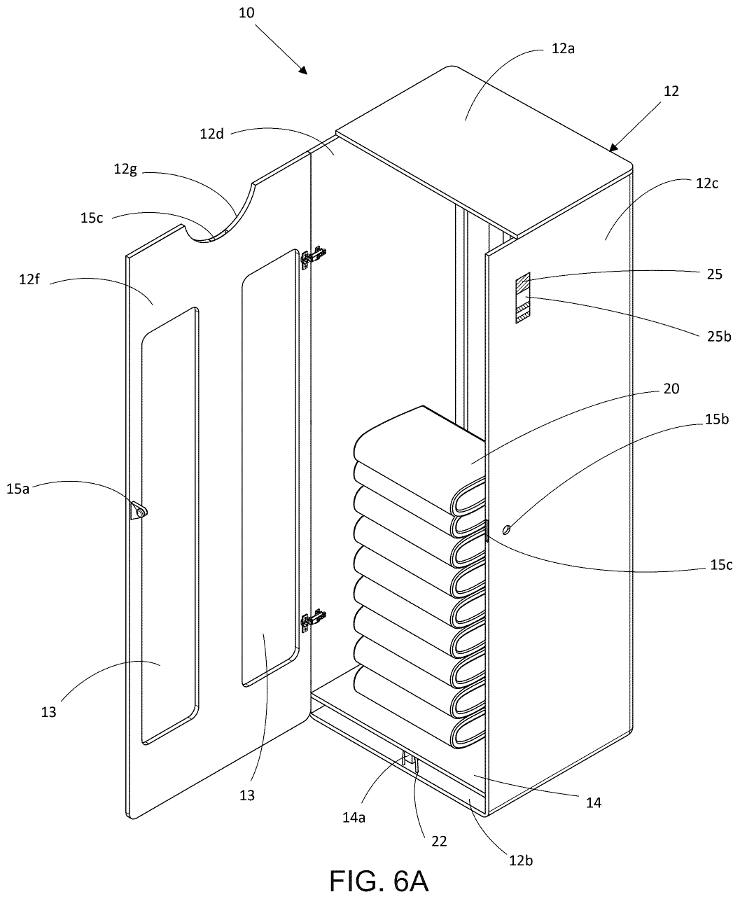

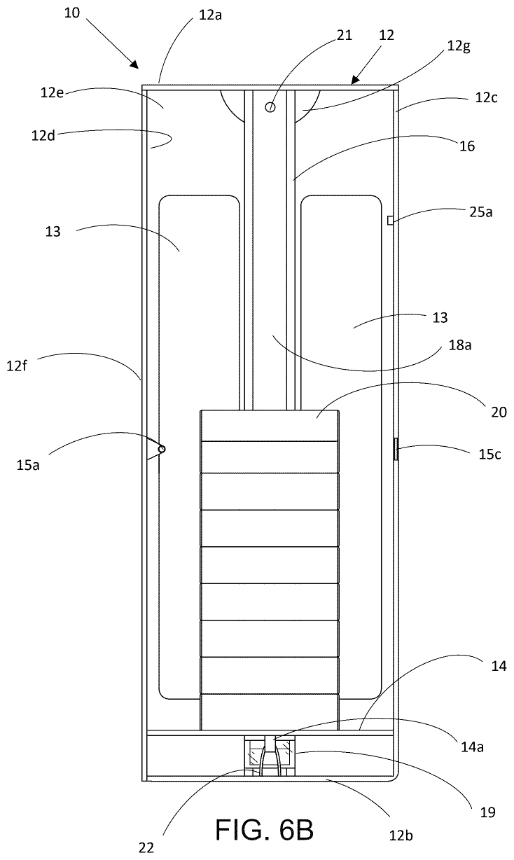

The door 12f is attached to side wall 12d by hinges 17, which permit pivotal opening and closing of the door. The door is further equipped with a male connector 15a configured for removable engagement with a corresponding female connector 15b provided on side wall 12c. This connector arrangement allows the door 12f to be securely closed, as shown in FIG. 1, and easily opened for access, as shown in FIG. 2. Additionally, a door slot 12g formed in the door 12f (FIGS. 1, 2, 6A) and a corresponding slot 12g formed in the rear wall 12e (FIGS. 3, 6B) create a semi-circular pathway that may interface with a lockable lid extension, as further described below with reference to FIGS. 10-12.

The lift platform assembly is provided within the interior space 12i of the housing 12 and is configured to support and elevate the stack of diapers 20 within the housing 12, ensuring that the uppermost diaper remains near the access opening 12h for convenient retrieval. The lift platform assembly comprises a vertically movable shelf platform 14, a single guide rail 16, and a spring mechanism operatively coupled to bias the platform 14 upward along the guide rails.

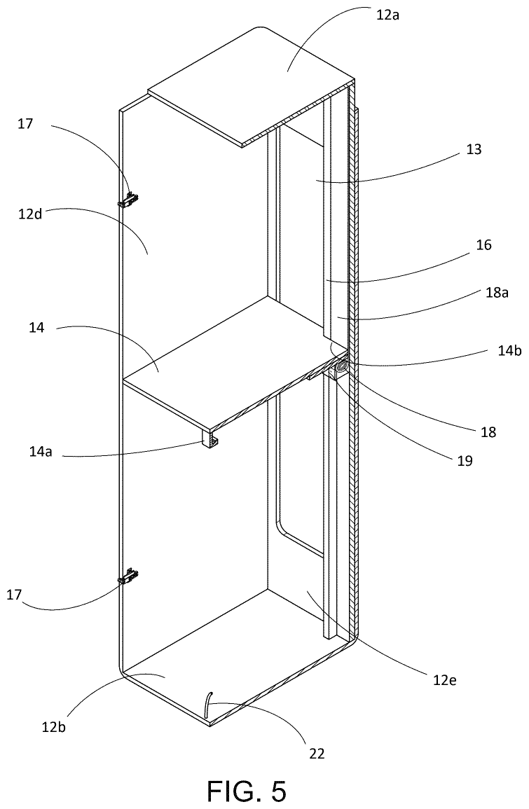

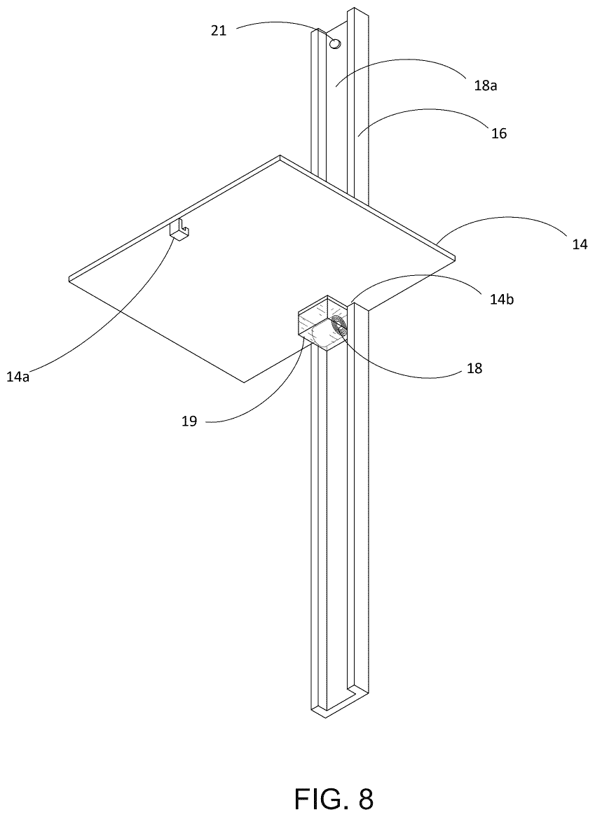

The platform 14 supports the diaper stack 20 (e.g., FIGS. 1, 2, 6A, 6B). The guide rail 16 is mounted to the rear wall 12e of the housing 12 using suitable fasteners. As shown in FIGS. 3, 5, 6b and particularly FIG. 8, in this embodiment the platform 14 is configured to move vertically along the single guide rail 16. The guide rail 16 maintains alignment and smooth vertical travel of the platform 14. As shown in FIGS. 5, 8, and 9, the platform 14 includes a notch 14b configured to receive the rail 16. The notch 14b is shaped to conform to the cross-sectional profile of the rail 16, which is generally C-shaped in this embodiment. This complementary geometry between the notch 14b and rail 16 allows the platform 14 to slide smoothly along the rail 16 during vertical movement, while maintaining proper alignment and lateral stability within the housing. The interaction between notch 14b and rail 16 facilitates guided travel of the platform 14 as it moves between the raised and lowered positions, contributing to the overall functionality and reliability of the lift platform assembly. The platform 14 also includes a hook 14a configured to engage with a loop connector 22 during the loading process as further described below for the loading mechanism.

The spring mechanism includes a coiled spring 18 that is disposed within a spring housing 19, which is mounted inside the interior of the housing 12. As illustrated in FIGS. 3, 6B, and 8, the spring 18 cooperates with the shelf platform 14 to provide a consistent upward biasing force that maintains the diaper stack near the access opening 12h. The spring 18 includes an extendable spring portion 18a with an upper end integrally connected to the rail 16 with a suitable fastener 21. The spring portion 18 is configured to unroll along the guide rail 16 when the shelf platform 14 is manually moved vertically downward during loading.

As shown in FIGS. 3, 5, 6A, 6B and 8, the spring housing 19, which contains the coiled spring 18, is mounted to a lower surface of the platform 14, namely, the surface opposite the upper surface of the platform 14 onto which the stack of diapers is placed. The notch 14b is configured and dimensioned not only to receive the guide rail 16 but also to permit the extendable spring portion 18a to pass through it. This arrangement allows the spring portion 18a to unroll along the guide rail 16 as the platform 14 is displaced downward during loading and to retract as the spring restores the platform 14 to its elevated position.

After loading, the tension in the spring causes the spring portion 18a to retract, effectively rolling the coil spring 18 back into the spring housing 19 as the platform 14 is gradually elevated upward along rail 16. It will be appreciated that the entire lift platform assembly, comprising the platform 14, spring coil 18, and rail 16, functions as a passive elevator mechanism, reducing manual effort and maintaining hygiene during repeated use.

The system is equipped with a user-friendly loading mechanism to facilitate secure and efficient reloading of diapers. Referring to FIGS. 2, 3, 6A and 6B, the loading mechanism comprises a loop connector 22 configured to temporarily engage with the hook 14a of the shelf platform 14. The loop connector 22 is securely mounted to the bottom wall 12b of the housing 12 in a manner that ensures its loop portion remains exposed and accessible, allowing it to be readily engaged with and disengaged from the hook 14a of the shelf platform 14. This configuration facilitates quick and reliable attachment of the platform to the base of the housing during reloading. The loop portion may protrude through an aperture or recessed opening in the bottom wall 12b, enabling the hook 14a of the platform to latch onto the loop connector 22 while diapers are being loaded. Once loading is complete, the platform is disengaged from the loop connector 22, allowing it to rise under the biasing force of the coil spring 18. The loop connector 22 may be mounted to the bottom wall 12b using mechanical fasteners such as rivets or screws, or alternatively, it may be integrally molded into a recessed or through-hole portion of the bottom wall 12b. Other suitable mounting methods may also be used, provided they ensure secure attachment and maintain the accessibility of the loop portion for engagement with the hook 14a of the shelf platform 14.

The system 10 is also equipped to provide real-time visual monitoring of the diaper supply. Specifically, it includes a visual indicator 25 comprising a vertically slidable indicator pin 25a that offers a clear, intuitive cue regarding the quantity of diapers remaining within the housing 12. The indicator pin 25a is positioned within a vertical guide channel and is visible through an external window or opening 25b, as illustrated in FIGS. 1 and 2.

As diapers are removed from the system, the shelf platform 14 gradually rises along the guide rail 16 under the upward biasing force of the spring coil 18. During this upward movement, the platform 14 engages the indicator pin 25a and pushes it upward within the guide channel. When the pin reaches its uppermost position, it provides a visual signal that the diaper supply is low or depleted. Conversely, when the unit is reloaded with a full stack of diapers, the platform 14 is caused to move downward, disengaging from the indicator pin and allowing it to descend to its lowermost position. This lowered position indicates that the system is fully or substantially stocked. Accordingly, the vertical position of the indicator pin 25a serves as a simple and effective visual gauge—where a raised pin signals a low or empty supply, and a lowered pin reflects a full or sufficient stock.

In addition to the indicator 25, the system 10 also features transparent windows 13 on the door 12f and rear wall 12e of the housing 12, enabling further real-time visual monitoring of the diaper quantity. These windows allow users to quickly assess the diaper supply without opening the housing, helping maintain hygiene and convenience. Optionally, transparent windows 13 may also be provided on the top wall 12a or side walls 12c and 12d to improve visibility from various angles.

Examples illustrating the process of loading and operating the dispensing system 10 are provided below.

To load the diaper dispensing system 10 with diapers, the user begins by opening the front-facing hinged door 12f to access the interior space 12i of the housing 12. Inside, the spring-biased lift platform assembly, comprising a vertically movable shelf platform 14 mounted to a guide rail 16 and biased upward by a coiled spring 18, must first be secured in the lowered position to allow for easy diaper loading. This is done by manually pressing the platform 14 downward along the guide rail 16, which causes the spring 18 to unroll and store potential energy. While holding the platform down, the user engages the hook 14a on the platform with the loop connector 22, which is mounted to the bottom wall 12b and exposed through a recessed or through-hole opening. This engagement temporarily locks the platform in place, neutralizing the upward spring force and creating a stable loading surface.

With the platform secured, the user can stack diapers 20 vertically onto the platform. As the diaper stack is loaded, the increased weight causes the platform 14 to remain fully lowered, which in turn disengages the indicator pin 25a from the platform. The pin, which is slidable within a guide channel and visible through an external opening 25b, descends to a lower position, visually signaling that the unit is fully or substantially stocked. Once loading is complete, the user releases the hook 14a from the loop connector 22, allowing the spring 18 to retract and gradually raise the platform 14. The platform then positions the top diaper near the diaper access opening 12h, formed by the top wall 12a and the partially open front door 12f, allowing for convenient, hygienic retrieval without fully opening the housing.

During use, each time a diaper is removed from the top of the stack, the platform 14 incrementally rises under the force of the spring 18, keeping the top diaper consistently positioned near the access opening 12h. As the platform ascends, it engages the indicator pin 25a, gradually pushing it upward along the guide channel. When the indicator pin reaches its uppermost position, it provides a clear visual cue (visible through the external opening 25b) that the diaper supply is low or depleted. In addition to this pin-based indicator, transparent windows 13 located on the door 12f and rear wall 12e of the housing 12 offer real-time visual access to the diaper stack, enabling users to quickly assess supply levels without opening the unit. These windows, which may also be optionally located on the top wall 12a or side walls 12c, 12d, are preferably constructed from durable, child-safe materials such as polycarbonate to ensure clarity, impact resistance, and safety.

Together, the lift platform assembly, loading mechanism, visual indicator 25, and transparent windows 13 provide an intuitive, hygienic, and user-friendly system for storing and dispensing diapers. The coordinated functionality of these components ensures that the system is easy to reload, offers continuous visual feedback on supply levels, and keeps diapers conveniently accessible throughout use.

FIGS. 10-12 illustrate a modified form of the diaper dispensing system 10 shown in FIGS. 1-9.

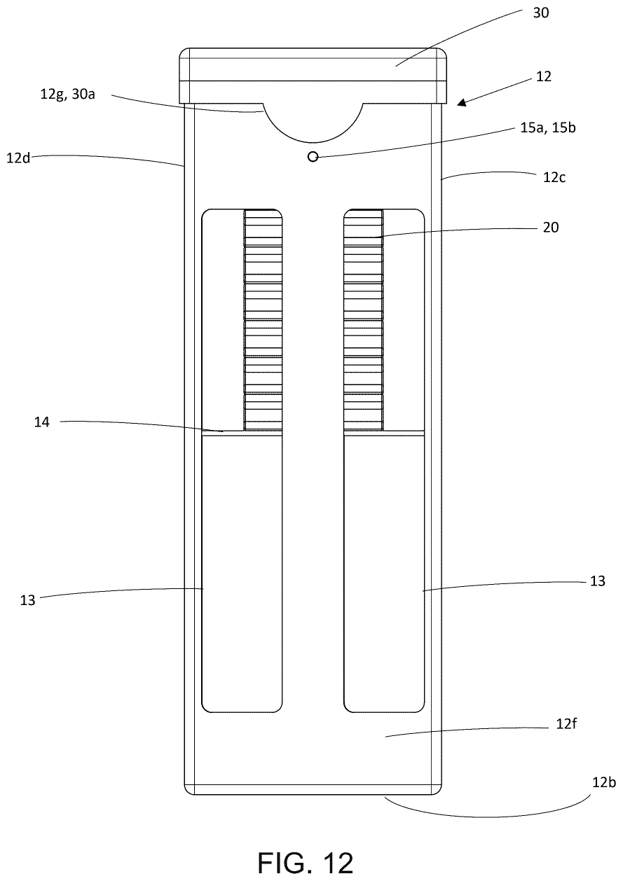

As shown in FIGS. 10-12, a lockable lid 30 is configured to be removably mounted to the top wall 12a of the housing 12 to further secure and enclose the diaper dispensing system 10. The lid 30 includes a pair of opposing semicircular extensions 30a that are designed to align with and nest within corresponding arcuate door slots 12g formed in the front-facing door 12f (FIGS. 1, 2, 6A) and the rear wall 12e (FIGS. 3, 6B). When the lid 30 is in the closed position, the extensions 30a mate with the door slots 12g to form a semi-circular pathway that completes a sealed upper enclosure, as illustrated in FIGS. 10 and 12. This configuration enhances the overall containment of the system while maintaining a streamlined and integrated appearance.

Each semicircular extension 30a includes a male connector 15a that is configured for releasable engagement with corresponding female connectors 15b provided on both the rear wall 12e and the front door 12f of the housing 12. These connectors help secure the lid 30 in the closed position, while also allowing for easy release and removal when access to the interior is needed.

The lid 30 further includes peripheral frame sections 30b that extend from the top edges of the side walls 12c and 12d and rear wall 12e. These frame sections provide a stable mounting surface to which the top wall 12a of the housing 12 is secured, and they also support the positioning and operation of the lid. The rear portion of lid 30 is pivotally attached to the frame section on the rear wall 12e using hinges 17, which permit the lid to open and close relative to the housing 12.

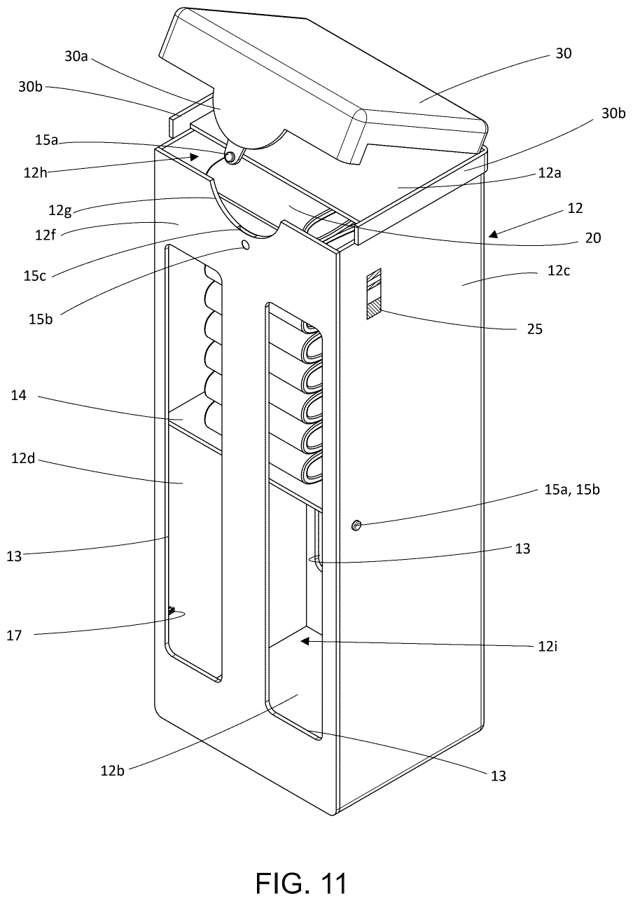

When closed, the lid 30 covers and seals the diaper access opening 12h, providing a tamper-resistant and hygienic barrier over the diaper stack. In this closed state, the system is fully enclosed and secured. When the lid 30 is opened, as shown in FIG. 11, the access opening 12h becomes exposed, allowing the user to load or retrieve diapers as needed. The combination of hinged functionality, connector-based locking, and integrated slot-and-extension design provides a secure, user-friendly, and visually coherent method for sealing and accessing the diaper dispensing system.

In addition to the embodiments described above, lid 30 may be mounted to the housing 12 using a variety of alternative mechanisms. For example, instead of hinged attachments, the lid may be secured using removable clips, sliding tracks, or interlocking tabs that allow the lid to be slid or snapped into position without the need for hinges. Magnetic closures embedded within the lid and the housing may also be used to maintain the lid in a closed position while allowing for easy detachment. Other mounting configurations may include flexible living hinges integrally formed with the lid, tension-based clasps, or tool-free twist-lock mechanisms. These alternative approaches may be selected based on manufacturing constraints, cost considerations, desired ease of use, or aesthetic preferences, while still achieving secure closure and accessibility of the diaper dispensing system.

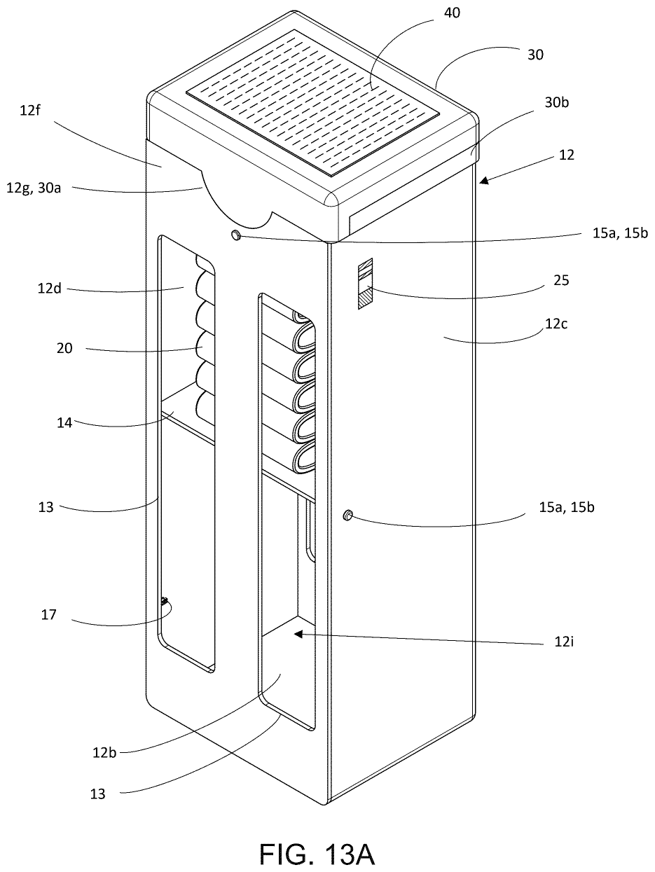

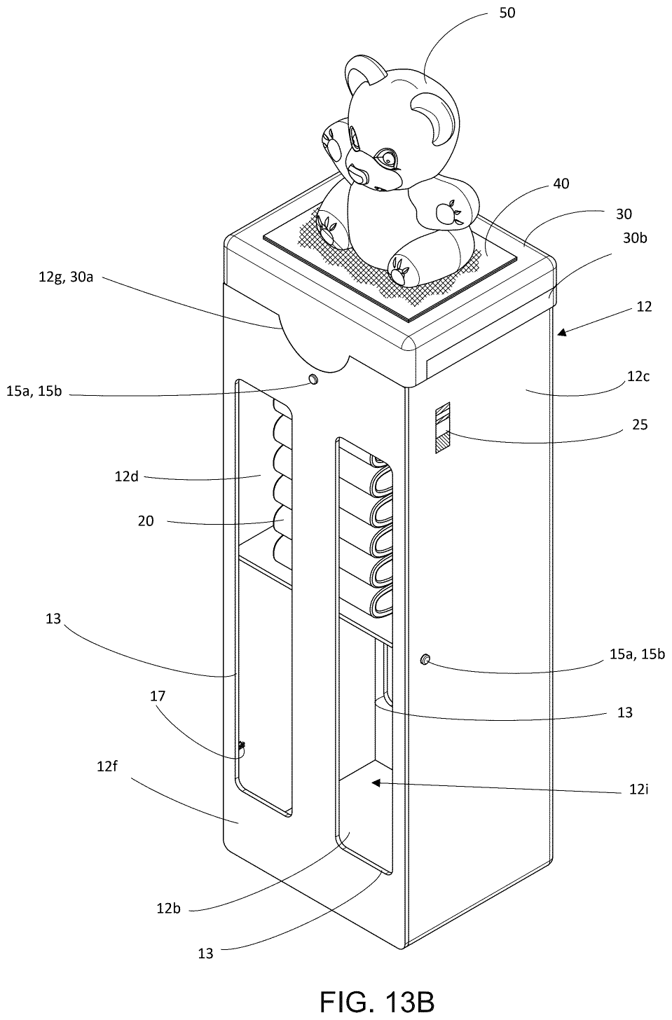

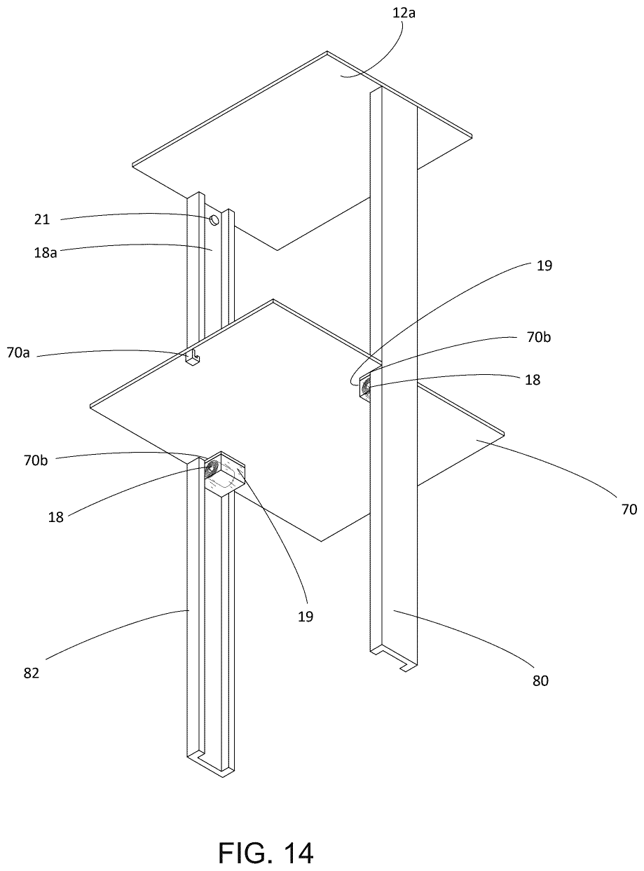

FIGS. 13-14 illustrate another modification to the diaper dispensing systems shown in FIGS. 1-9 and 10-12.

As shown in FIGS. 13A-13B, a connecting element 40 is secured to the top surface of the lockable lid 30 of the diaper dispensing system 10. This element 40 enables attachment of a decorative top 50, allowing aesthetic customization of the system to complement various nursery themes or personal preferences. The decorative top 50 can be removably connected to the element 40 using hook-and-loop fasteners, adhesive pads, or other suitable means that allow for easy attachment and removal. When the lid 30 is not used, the connecting element 40 may alternatively be secured directly to the top wall 12a of the housing 12, enabling similar decorative customization in alternative configurations.

In the illustrated embodiment, the decorative top 50 is in the form or appearance of a teddy bear. However, other suitable decorative elements include animal figurines (e.g., elephant, bunny, giraffe), alphabet blocks, cartoon characters, seasonal ornaments (e.g., snowman or pumpkin), or monogrammed initials. This customization feature enhances the visual appeal of the diaper dispensing system 10, allowing it to blend harmoniously with nursery decor or serve as a playful and engaging element for the child. The removable nature of the decorative top also enables easy updates or theme changes without affecting the system's functionality.

In alternative embodiments, the connecting element 40 and decorative top 50 may be mounted using various attachment mechanisms other than hook-and-loop fasteners or adhesive pads. For example, the connecting element 40 may include an integrated threaded post or bayonet-style connector that allows the decorative top 50 to be twisted or snapped into place securely. Magnetic attachments may also be employed to provide tool-free and easily reversible mounting of the decorative top. Additionally, the decorative top 50 may be mounted via sliding grooves, locking tabs, suction cups, or flexible clips that engage with corresponding features on the connecting element 40. These alternative mounting approaches may be selected based on user preference, durability requirements, case of manufacturing, or safety considerations, while still allowing for removable and interchangeable decorative customization.

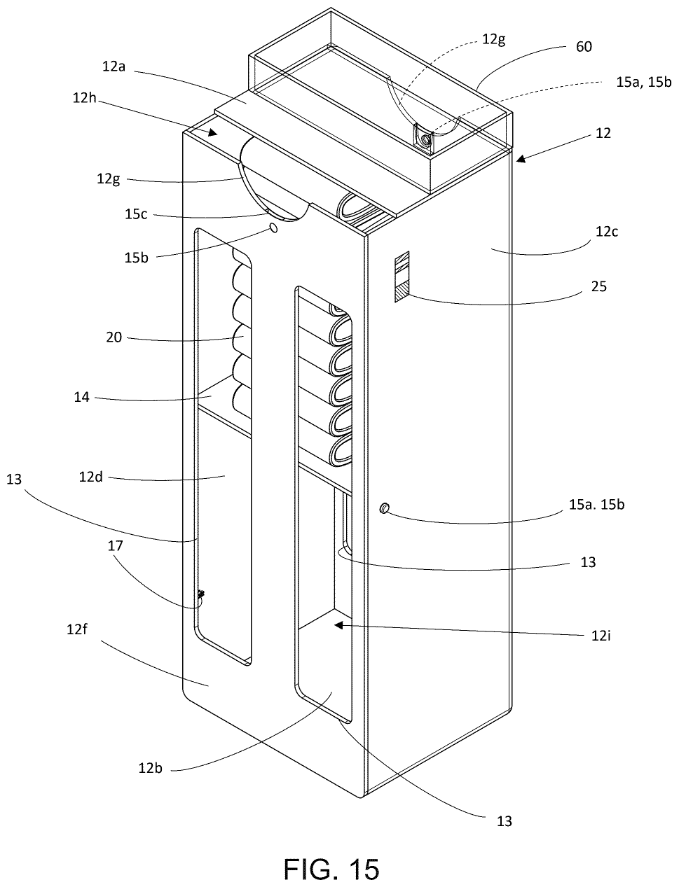

FIG. 15 illustrates another modification of the diaper dispensing system shown in FIGS. 1-9, 10-12, and 13A-13B. In this modified embodiment, a wipe holder 60 is removably mounted to the top surface 12a of the housing 12. The wipe holder 60 is secured using a locking mechanism, such as mating male and female connectors 15a and 15b, similar to those described above in connection with the lockable lid 30 of FIGS. 10-12. This accessory enhances the utility of the system by integrating a convenient storage location for baby wipes, thereby transforming the unit into a multi-functional diapering station. When the lid 30 is present, the wipe holder 60 can alternatively be mounted to the top surface of the lid, maintaining easy access while preserving the aesthetic and functional configuration of the system.

In alternative embodiments, the wipe holder 60 may be mounted to the top surface 12a of the housing 12 or to the lid 30 using a variety of securing methods other than mating male and female connectors. For example, the holder may be attached using magnetic bases, adhesive-backed mounting pads, suction mechanisms, or snap-fit features that engage with recesses or projections on the mounting surface. Additional options include clamp-based attachments, sliding rails or tracks, and interlocking flange systems that allow for tool-free installation and removal. These alternative mounting methods provide flexibility in design and may be selected based on desired ease of use, stability, or manufacturing simplicity, while still maintaining the functionality and convenience of an integrated wipe storage accessory.

FIG. 14 illustrates another embodiment of the diaper dispensing system according to the present invention.

In the embodiment shown in FIG. 14, the shelf platform 70, provided with a hook 70a and notches 70b, is operatively supported between two guide rails 80 and 82, which are mounted vertically along the right and left interior side walls 12c and 12d of the housing 12. The platform 70 is configured to travel vertically along these rails, guided by its engagement with the rails and supported by the notches 70b which are formed along its lateral edges. The notches 70b are configured and function in the same manner as notch 14b described above in connection with the embodiment of FIGS. 1-9. Specifically, the notches 70b allow the platform 70 to move smoothly and in alignment with the guide rails while maintaining clearance for the spring components.

Each of the guide rails 80 and 82 is paired with a corresponding spring coil 18, housed within the spring housings 19 and positioned to apply an upward biasing force to the platform 70. The spring coils 18 function similarly to the single-spring embodiment described earlier: when the platform 70 is manually depressed to load diapers, the spring coils 18 extend and store mechanical energy. Upon release of the platform, typically after engaging and then disengaging the hook 70a from the loop connector 22, the stored energy in the spring coils causes them to retract, gradually lifting the platform upward along the guide rails.

As diapers are removed during use, the spring coils 18 continue to exert upward pressure, maintaining the top diaper near the access opening 12h. The dual-spring arrangement ensures that the platform rises evenly on both sides, reducing tilt, wobble, or uneven movement, and providing stable, balanced elevation of the diaper stack throughout operation. This configuration is particularly advantageous for maintaining consistent access to the diapers and for supporting heavier or unevenly stacked loads with improved structural control and reliability.

The dispensing system 10 may be constructed from a variety of durable and child-safe materials selected to balance functionality, aesthetics, and manufacturing considerations.

The housing 12, including the top wall 12a, bottom wall 12b, side walls 12c and 12d, rear wall 12e, and front-facing door 12f, may be formed from rigid materials such as injection-molded plastic, wood, synthetic wood (e.g., MDF or composite), or metal (e.g., aluminum or coated steel). These materials offer structural integrity and can be selected based on cost, weight, appearance, and intended use environment (e.g., home or commercial nursery settings).

The transparent windows 13, located on the door 12f and/or other walls of the housing, may be constructed from polycarbonate, which provides excellent clarity, impact resistance, and long-term durability while being safe for use around children. Alternative transparent materials may include acrylic or child-safe, shatter-resistant plastics, provided they meet the required safety and performance standards.

The shelf platforms, including platform 14 in the embodiment of FIGS. 1-9 and platform 70 in the embodiment of FIG. 14, may be formed from rigid plastic, lightweight metal (such as aluminum), or durable wood composites. These materials should be strong enough to support a full stack of diapers while allowing for smooth movement along the guide rails. Plastics used for the platforms may include ABS or polypropylene, which are known for their strength and resistance to deformation.

The guide rails, rail 16 in the single-rail embodiment and rails 80 and 82 in the dual-rail embodiment, may be constructed from metal to ensure structural stability and long-term durability under repeated use. Suitable metals include stainless steel, anodized aluminum, or powder-coated steel, which resist corrosion and wear while supporting the guided vertical movement of the shelf platform.

The spring mechanisms 18 used in each embodiment are preferably spiral coil springs or clock springs housed within spring housings 19. These springs, which are a type of constant force spring, are selected for their ability to store and release mechanical energy smoothly and consistently over repeated cycles. The springs may be formed from tempered steel, phosphor bronze, or stainless steel, depending on the desired balance between strength, fatigue resistance, and environmental durability.

Other components, such as the loop connector 22, hooks 14a and 70a, and various hinges 17, connectors 15a/15b, and indicator pins 25a, may be constructed from molded plastic, die-cast metal, or reinforced polymer composites, chosen based on their required mechanical function, frequency of use, and safety considerations.

Overall, the selection of materials across the system is intended to ensure a lightweight, durable, hygienic, and child-safe product capable of withstanding regular use while offering customizable aesthetic options to suit various user preferences and nursery settings.

The dispensing system of the present invention may be manufactured in various dimensions and configurations to accommodate different environments, user preferences, or storage requirements. While the figures illustrate specific shapes, configurations and component arrangements, it is understood that the invention is not limited to precise dimensions or the structural features depicted. For example, the housing may be taller, wider, or more compact depending on the intended location of use, such as a home nursery, daycare facility, or healthcare setting. Similarly, the number and orientation of guide rails, coil springs, access doors, platforms, or windows may be modified without departing from the scope and spirit of the invention. Such variations are contemplated and fall within the broad teachings provided herein.

The dispensing system described according to the embodiments of the present invention described above offers numerous advantages that enhance the diaper-changing experience for parents and caregivers. By incorporating a spring-biased lift platform assembly, the system maintains the top diaper at an accessible height, reducing the need for awkward reaching and minimizing disruption during use. This lift platform assembly ensures consistent and effortless elevation of the diaper stack as diapers are removed, improving convenience and speeding up the diaper-changing process. Additionally, it helps prevent diapers from becoming compressed or damaged, preserving their shape and usability.

The lift platform assembly can be configured with either a single rail or dual rails, each providing distinct benefits: a single rail design offers simplicity, ease of manufacturing, and smooth vertical movement, while a dual-rail configuration enhances stability and balanced support, preventing tilt or jamming during operation. The loading mechanism further enhances usability by enabling quick and easy reloading of the diaper stack, minimizing mess and disruption during refills. This mechanism helps maintain cleanliness and hygiene by reducing contact with the stored diapers and simplifying the replenishment process.

The hinged door with a partial access opening allows for quick retrieval of diapers while limiting exposure to contaminants, thereby promoting hygiene and cleanliness. The secure yet easily operable male-female connector ensures the door remains closed during storage but can be swiftly opened when needed. Furthermore, the inclusion of door and rear wall slots forming a semi-circular pathway provides flexibility for integrating lockable lid extensions, adding an extra layer of security and convenience. Overall, the system's modular design, ergonomic features, and hygienic access points combine to deliver a safe, sanitary, and user-friendly diaper dispensing solution.

Unlike conventional dispensing systems with exposed or unsecured spring components, the system according to the embodiments of the present invention features a fully enclosed spring mechanism that is safely integrated within the housing to eliminate potential contact with moving or pressurized parts. This design not only improves the structural integrity and reliability of the dispensing function, but also significantly reduces the risk of accidental injury, particularly in homes, daycare facilities, or other environments where young children may interact with the device. To further enhance safety, the system is particularly suitable for use with child-resistant access doors equipped with locking connectors which ensure that the door remains securely closed during normal use and cannot be easily opened by an unsupervised child. These features collectively address a major drawback in the prior art by providing a dispensing system that is both functionally effective and specifically engineered to minimize safety hazards in everyday use.

The embodiments of the dispensing system featuring a decorative top and integrated wipe holder offers enhanced functionality and aesthetic appeal. The decorative top allows the system to blend seamlessly with nursery or home décor, making it an attractive addition to any room while maintaining its practical purpose. Meanwhile, the built-in wipe holder provides convenient, easy access to wipes during diaper changes, streamlining the process and reducing the need to carry separate wipe containers. This integration promotes organization, saves space, and ensures that both diapers and wipes are readily available in one compact, user-friendly unit, thereby improving overall efficiency and convenience for parents and caregivers.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A storage and dispensing system comprising:
a housing defining an interior space configured to receive and store one or more items;
a lift platform assembly disposed within the housing and configured to move vertically to support and elevate the one or more items within the interior space; and
a loading mechanism operatively connected to the lift platform assembly and configured to facilitate loading or placement of the one or more items onto the lift platform assembly;
wherein the lift platform assembly is movable between a lowered position and a raised position to enable selective dispensing of the one or more items from the housing, and
wherein the loading mechanism is configured to interact with the lift platform assembly to load the one or more items for dispensing.

2. The system of claim 1, wherein the housing comprises a top wall, a bottom wall, opposing side walls, a rear wall, and a door configured to be opened for providing access into the interior space of the housing; and wherein the top wall and the door define a diaper access opening configured to allow a user to retrieve an item from the housing without fully opening the door.

3. The system of claim 1, wherein the lift platform assembly comprises a vertically movable shelf platform, at least one guide rail, and at least one spring mechanism operatively coupled to bias the shelf platform upward along the guide rail.

4. The system of claim 3, wherein the at least one guide rail comprises a single guide rail secured to the rear wall of the housing, the guide rail being configured to maintain alignment and facilitate smooth vertical movement of the shelf platform.

5. The system of claim 3, wherein the spring mechanism comprises a single coiled spring disposed within a spring housing and having an extendable spring portion with an upper end connected to the guide rail.

6. The system of claim 5, wherein the extendable spring portion is configured to unroll along the guide rail when the shelf platform is moved downward during loading of the one or more items for dispensing, and to retract into the spring housing to elevate the shelf platform after loading the one or more items for dispensing.

7. The system of claim 6, wherein the shelf platform includes a notch configured to receive the guide rail, the notch having a profile shaped to conform to the cross-sectional shape of the guide rail to enable smooth and aligned vertical movement of the shelf platform along the guide rail.

8. The system of claim 7, wherein the spring housing is mounted to a surface of the shelf platform opposite to a surface of the shelf platform configured to support the one or more items; and wherein the notch is further configured and dimensioned to permit passage of the spring portion of the spring mechanism through the notch to allow the spring portion to unroll along the guide rail during downward displacement of the platform and retract during upward return movement.

9. The system of claim 3, further comprising a visual indicator including an indicator pin configured to be engaged by the shelf platform during upward movement to provide a visual cue indicative of a low or depleted supply of items.

10. The system of claim 1, wherein the housing comprises a door configured to be opened for providing access into the interior space of the housing; and wherein the housing includes one or more transparent windows positioned on at least one of the door and a wall of the housing, the one or more transparent windows being configured to allow visual inspection of the quantity of items stored within the interior space of housing without opening the door.

11. The system of claim 1, wherein the loading mechanism comprises a loop connector mounted to a bottom wall of the housing, and a shelf platform onto which the one or more items are loaded, the shelf platform having a hook member configured to engage with the loop connector during loading of the one or more items onto the shelf platform.

12. The system of claim 1, further comprising a lockable lid removably mounted to a top wall of the housing; wherein the lid includes a pair of opposing semicircular extensions configured to align with corresponding arcuate slots formed in a door and a rear wall of the housing, such that, when the lid is in a closed position, the semicircular extensions mate with the slots to form a sealed upper enclosure.

13. The system of claim 12, further comprising a decorative top configured to be removably attached to a connecting element secured to either a top surface of the lockable lid or directly to a top wall of the housing.

14. The system of claim 1, further comprising a wipe holder removably mounted to a top wall of the housing.

15. The system of claim 1, wherein the lift platform assembly comprises a shelf platform supported between two vertically oriented guide rails mounted along opposing interior side walls of the housing, and two coil springs each housed within a respective spring housing and paired with a corresponding guide rail, the shelf platform being configured to move vertically along the guide rails under the influence of the coil springs.

16. The system of claim 15, wherein the shelf platform includes notches formed along its lateral edges, the notches being shaped and positioned to engage the guide rails to allow smooth, aligned vertical movement of the shelf platform while maintaining clearance for components of the two coil springs.

17. The system of claim 15, wherein the spring coils are configured to apply upward biasing forces to the shelf platform, wherein manual downward displacement of the platform causes the spring coils to extend and store mechanical energy, and subsequent release of the platform causes the spring coils to retract and lift the platform upward along the guide rails.

18. A storage and dispensing system comprising:
a housing defining an interior space configured to receive and store one or more items;
a lift platform assembly disposed within the housing and configured to support and elevate the one or more items within the interior space, the lift platform assembly comprising:
a shelf platform configured to move vertically within the housing;
at least one guide rail secured to an interior surface of the housing and configured to guide the vertical movement of the shelf platform; and
at least one coil spring mechanism operatively coupled to the shelf platform and configured to exert an upward biasing force to elevate the shelf platform while the at least one guide rail guides the vertical movement of the shelf platform; and
a loading mechanism operatively associated with the lift platform assembly and configured to temporarily restrain the shelf platform in a lowered position during placement of the one or more items onto the shelf platform, and to release the shelf platform to enable elevation by the at least one coil spring mechanism,
wherein the shelf platform is configured to move downward during loading to extend the at least one coil spring mechanism and store mechanical energy, and is biased upward by the release of the stored mechanical energy in the at least one coil spring mechanism during dispensing.

19. The system of claim 18, wherein the at least one guide rail comprises two vertically oriented guide rails mounted along opposing interior side walls of the housing, and two coil springs each housed within a respective spring housing and paired with a corresponding guide rail, the shelf platform being supported between the two guide rails and being configured to move vertically along the guide rails under the influence of the two coil springs.

20. The system of claim 19, wherein the shelf platform includes notches formed along its lateral edges, the notches being shaped and positioned to engage the guide rails to allow smooth, aligned vertical movement of the shelf platform while maintaining clearance for components of the two coil springs; and wherein the spring coils are configured to apply upward biasing forces to the shelf platform, wherein manual downward displacement of the platform causes the spring coils to extend and store mechanical energy, and subsequent release of the platform causes the spring coils to retract and lift the platform upward along the guide rails.
